(19) United States
(12) Patent Application Publication
(10) Pub. No.: US 2012/0128644 A1
(43) Pub. Date: May 24, 2012

(54) Use of Bacteria to Treat and Prevent Respiratory Infections

(75) Inventor: Jeffrey D. Hillman, Gainesville, FL (US)

(73) Assignee: Oragenics, Inc.

(21) Appl. No.: 13/302,006

(22) Filed: Nov. 22, 2011

Related U.S. Application Data
(60) Provisional application No. 61/417,011, filed on Nov. 24, 2010.

(51) Int. Cl. A61K 35/74; A61P 31/12; A61P 31/10; A61P 31/04; A61P 11/00; A61P 31/00

(52) U.S. Cl. 424/93.44; 424/93.45; 424/93.4

(57) ABSTRACT

The invention provides methods for treating respiratory infections by administering compositions comprising one or more isolated, non-pathogenic, hydrogen peroxide-producing bacterial species or strains to the mouth, oropharynx, larynx, nasal cavity, fauces of a subject.

### PRIORITY

This application claims the benefit of U.S. Ser. No. 61/417,011, filed Nov. 24, 2010, which is incorporated herein by reference in its entirety.

### BACKGROUND OF THE INVENTION

Upper and lower respiratory infections are very common and cause substantial illness and billions of dollars of economic loss every year. Most pathogens that cause respiratory infections are spread through the air and/or through direct contact. There is a need in the art for effective and easy to use compositions to treat and prevent respiratory infections and their symptoms.

### BRIEF SUMMARY OF THE INVENTION

One embodiment of the invention provides a method for treating a respiratory infection. The method comprises administering a composition comprising one or more isolated, non-pathogenic, hydrogen peroxide-producing bacterial species or strains to mouth, oropharynx, larynx, nasal cavity, fauces or combination thereof to a subject in need thereof. The one or more isolated, non-pathogenic, hydrogen peroxide-producing bacterial species or strains can be Lactobacillus, Bifidobacteria, viridans Streptococcus, Leuconostoc, Pediococcus, Lactococcus, or combinations thereof. The one or more isolated, non-pathogenic, hydrogen peroxide-producing bacterial species or strains can be (a) one or more isolated Streptococcus oralis strains, or (b) one or more isolated strains of Streptococcus uberis, or (c) a combination thereof. The composition can be not swallowed and/or not substantially delivered to the gastrointestinal tract. Alternatively, the composition can be swallowed. The composition can be administered to the subject up to four times a day, about once a week, or about once a month. The respiratory infection can be reduced in severity or eliminated, the symptoms of the respiratory infection can be eliminated or reduced in severity or number, the duration of the respiratory infection can be reduced in length of time, or a combination thereof. The composition can further comprise one or more lactate dehydrogenase-deficient mutans Streptococcus, Lactobacillus, Bifidobacteria, viridans Streptococcus, Leuconostoc, Pediococcus, or Lactococcus species or strains. The one or more lactate dehydrogenase-deficient mutans Streptococcus species can comprise Streptococcus rattus. The subject can have an increased susceptibility to a respiratory infection.

Another embodiment of the invention provides a method of reducing the amount in a subject of bacteria, fungi, or viruses that can cause a respiratory infection in a subject. The method comprises administering a composition comprising one or more isolated, non-pathogenic, hydrogen peroxide-producing bacterial species or strains to a mouth, oropharynx, fauces, larynx or nasal cavity or a combination thereof to a subject having one or more strains or species of bacteria, viruses or fungi that can cause a respiratory infection in their mouth, oropharynx, fauces, larynx or nasal cavity. The composition can further comprise one or more lactate dehydrogenase-deficient mutans Streptococcus, Lactobacillus, Bifidobacteria, viridans Streptococcus, Leuconostoc, Pediococcus, or Lactococcus species or strains. Optionally, prior to the administration, one or more bacteria, viruses, or fungi that can cause a respiratory infection can be detected in the subject. Optionally, prior to the administration of the composition, the subject can be diagnosed with a respiratory infection caused by one or more bacteria, viruses, or fungi.

Even another embodiment of the invention provides a method of preventing a respiratory infection or one or more symptoms of a respiratory infection in a subject having an increased susceptibility to a respiratory infection. The method comprises administering a composition comprising one or more isolated, non-pathogenic, hydrogen peroxide-producing bacterial species or strains to a mouth, oropharynx, fauces, larynx, nasal cavity, or combination thereof to the subject. The composition can further comprise one or more lactate dehydrogenase-deficient mutans Streptococcus, Lactobacillus, Bifidobacteria, viridans Streptococcus, Leuconostoc, Pediococcus, or Lactococcus species or strains. The subject can have cystic fibrosis, asthma, cystic fibrosis, emphysema, mesothelioma, chronic obstructive pulmonary disorder, acute respiratory distress syndrome, obstructive lung disease, chronic obstructive pulmonary disease, restrictive lung disease, malignant or benign tumors of the respiratory system. The subject can be a smoker or an infant or a child.

Still another embodiment of the invention provides a method of preventing a respiratory infection or one or more symptoms of a respiratory infection in a subject. The method comprises: (a) obtaining data regarding an therapeutically effective dosage range for prevention of one or more respiratory infection symptoms in a particular type of subject and determining the therapeutically effective dosage range for the particular type of subject; and (b) administering the therapeutically effective dosage range for the particular type of subject of one or more isolated, non-pathogenic, hydrogen peroxide-producing bacterial species or strains to a mouth, oropharynx, larynx, nasal cavity, fauces, or combination thereof to the particular type of subject. The particular type of subject can be a subject with cystic fibrosis, an immune deficiency, asthma, cystic fibrosis, emphysema, mesothelioma, chronic obstructive pulmonary disorder, acute respiratory distress syndrome, obstructive lung disease, chronic obstructive pulmonary disease, restrictive lung disease, malignant or benign tumors of the respiratory system. The particular type of subject can be a smoker, or an infant, a child, an adult or elderly. The composition can further comprise one or more lactate dehydrogenase-deficient mutans Streptococcus, Lactobacillus, Bifidobacteria, viridans Streptococcus, Leuconostoc, Pediococcus, or Lactococcus species or strains.

### DETAILED DESCRIPTION OF THE INVENTION

As used herein, the singular forms “a,” “an”, and “the” include plural referents unless the context clearly dictates otherwise.

Methods of the invention comprise administering a composition comprising certain bacteria or combinations of bacteria to a host to treat or prevent respiratory infections. A composition of the invention comprises one or more isolated, non-pathogenic, hydrogen peroxide-producing species or strains of bacteria.

While not wishing to be bound to any particular theory, it is believed that the bacterial compositions of the invention (1) produce an amount of H₂O₂ that prevents or inhibits viral, bacterial and/or fungal growth, colonization, or infection and/or (2) reduce and/or prevent the ability of viruses, bacteria, or fungi to attach to, infect, or colonize cells/surfaces of the host. The bacteria of the invention can, for example, outcompete pathogenic bacteria, viruses and fungi, block attachment of pathogenic bacteria, viruses, and fungi to host surfaces, including cell surfaces, block attachment of pathogenic bacteria, viruses and fungi to host binding sites, or block attachment of pathogenic bacteria, viruses and fungi to host cell receptors used by pathogenic bacteria, viruses or fungi to colonize or infect the host. By “outcompete” is meant that the bacterial compositions of the invention have a slight, modest, or strong competitive advantage or edge over the pathogenic bacteria, viruses or fungi, such that they will grow, live, colonize, or exist in a host such that the pathogenic bacteria, viruses or fungi are excluded or reduced in number. The result is the prevention or amelioration of a disease, for example a respiratory infection.

In one embodiment of the invention the bacteria of the invention do not induce a significant immune response in the host and/or do not augment an immune response in a host. In one embodiment of the invention the induction of a significant immune response in the host is not the principle mechanism for treatment and/or prevention of respiratory infections.

Respiratory infections include, for example, upper respiratory infections, lower respiratory infections, sinus infections, pharyngeal infections, laryngitis, influenza, pneumonia, nasopharyngitis, atopic dermatitis, tracheitis, laryngopharyngitis, laryngotracheitis, rhinitis, bronchitis, bronchopneumonia, ear infections, tonsillitis or combinations thereof.

### Upper Respiratory Infections

Many different viruses, bacteria, and fungi can cause upper and lower respiratory infections and associated symptoms. Over 200 different viruses can cause upper and lower respiratory infections, including, for example, rhinovirus, coronavirus, parainfluenza virus, adenovirus, enterovirus, respiratory syncytial virus (RSV), bocavirus, influenza viruses, human metapneumovirus (hMPV), orthomyxoviridae, cytomegalovirus, Epstein-Barr virus, herpes simplex virus, and morbillivirus.

Bacteria responsible for upper and lower respiratory infections include, for example, group A Streptococcus (Streptococcus pyogenes), Haemophilus influenzae, Psuedomonas spp., Mycobacteria spp., Pasterurella spp., Pneumocystis jiroveci, Mycobacterium tuberculosis, Peptostreptococcus spp., Fusobacterium prevotella, Klebsiella pneumonia, Moraxella catarrhalis, Streptococcus pneumoniae, Chlamydophila pneumoniae, Mycoplasma pneumoniae, Legionella pneumophila, Staphylococcus aureus, Corynebacterium diphtheriae, Neisseria gonorrhoeae, Fusobacterium necrophorum, Bordetella pertussis, Treponema pallidum, Chlamydia trachomatis, Pseudomonas aeruginosa, Bacillus anthracis and Chlamydophila psittaci.

Fungi that can cause upper and lower respiratory infections include, for example, Histoplasma capsulatum, Coccidioides immitis, Blastomyces dermatitidis, Aspergillus spp., Candida spp., Candida albicans, Cryptococcus neoformans, Zygomycetes spp., Fusarium spp., Penicillium marneffii, Pseudallescheria boydii, Phialemonium obovatum, Pythium insidiosum, Absidia corymbifera, Enterocytozoon bieneus, Hormographiella aspergillata, Aspergillis spp., Histoplasma capsulatum, Pneumocystis jiroeci, Curvularia spp., Bipolaris spp., Exserohilum spp., Mucor spp., Rhizopus spp., Absidia spp., Cunninghamella spp., Metarrhizium anisopliae and Irpex lacteus.

Upper respiratory infections are infections of the nose, sinuses, nasal cavity, pharynx or larynx and include, for example, tonsillitis, pharyngitis, rhinitis, rhinosinusitis, nasopharyngitis, laryngitis, sinusitis, laryngopharyngitis, laryngotraceheitis, larynepiglottitis, laryngotracheitis, tracheitis, otitis media, and the common cold.

Symptoms of upper respiratory infections include, for example, cough, itchy, watery eyes, nasal discharge, sore throat, nasal congestion, headache, fever, facial pressure, pain in swallowing, headache, malaise, fatigue, chills, body ache, weakness, myalgia, pain and pressure of the ear, and sneezing.

### Sinus Infections

Sinus infections cause the inflammation of paranasal sinuses. Acute sinusitis can be brought on by other upper respiratory tract infections. Chronic sinusitis lasts longer than three months. Chronic and acute sinusitis can be caused by, for example, the viruses, bacteria, and fungi that cause respiratory infections as listed above.

The symptoms of chronic and acute sinusitus include, for example, nasal congestion, facial pain, headache, coughing, an increase in asthma symptoms, malaise, thick green or yellow discharge, facial discomfort, pain and pressure, dizziness, toothache, anosmia, and halitosis.

### Pharyngeal Infections

Pharyngitis (acute and chronic), pharyngotonsillitis, and nasopharyngitis cause inflammation of the throat and/or pharynx. The symptoms of pharyngeal infections include cold-like symptoms, sore throat, enlarged or inflamed tonsils, redness and swelling of the throat, lymph node enlargement, difficulty in swallowing or breathing, fever, headache, generalized aches, and cough. Other pharyngeal infections include peritonsillar abscesses, submandibular space infection, and epiglottitis.

Pharyngeal infections can be caused by, for example, viruses, bacteria, and fungi that cause respiratory infections as listed above.

### Ear Infections

Ear infections include, for example, otitis externa (chronic and acute), otitis media, secretory otitis media, chronic suppurative otitis media, otitis interna, and otomycosis. Symptoms of ear infections include, for example, pain in the ear and jaw, vertigo, hearing loss or impairment, pruritus, inflammation, scaling, tinnitus, fever, exudative inflammation, rigor, meningism, sensitive mastoid process, pus and ear exudate, and discomfort.

Bacteria, viruses and fungi that can cause ear infections include, for example, those listed above as causes of respiratory infections.

### Tonsillitis

Tonsillitis is the inflammation of the tonsils. Symptoms include red or swollen tonsils, tender, stiff, and/or swollen neck, bad breath, sore throat, painful or difficult swallowing, cough, headache, sore eyes, body aches, fever, chills, nasal congestion, ear pain, fever, rheumatic fever, glomerulonephritis, swollen lymph nodes in the neck or combinations thereof. Tonsillitis can be caused by the viruses, bacteria, and fungi that cause respiratory infections as listed above.

### Lower Respiratory Infection

The lower respiratory tract is the part of the respiratory tract below the vocal cords and includes the trachea, primary bronchi, and lungs. Lower respiratory infections include, for example, pneumonia, influenza, lung abscesses, and bronchitis (acute and chronic). Symptoms of lower respiratory infections include, for example, shortness of breath, weakness, fever, chest congestion, coughing and fatigue. Lower respiratory infections can be caused by the viruses, bacteria, and fungi that cause respiratory infections as listed above.

### Compositions of the Invention

The invention provides for using a composition comprising one or more non-pathogenic, hydrogen peroxide-producing viridans Streptococci species or strains, and/or one or more non-pathogenic, hydrogen peroxide-producing Lactobacillus species or strains and/or one or more non-pathogenic, hydrogen peroxide-producing Bifidobacteria species or strains and/or one or more non-pathogenic, hydrogen peroxide producing Lactococcus species or strains and/or one or more non-pathogenic, hydrogen peroxide producing Pediococcus species or strains and/or one or more non-pathogenic, hydrogen peroxide producing Leuconostoc species or strains. A non-pathogenic organism does not cause disease in a healthy host and does not cause harm to a healthy host. In one embodiment of the invention the bacterial strains can be safe for administration to humans and other mammals, and can optionally be generally recognized as safe. The bacterial strains of the invention can attach or adhere to a mouth surface (including the oral cavity, mucosa, glands, teeth and tongue), oropharynx surface, larynx, nasal cavity, and/or fauces surface by virtue of electrostatic interactions, van der Waals interactions, other interactions, or protein or polysaccharide adhesins on the bacterial surface that recognize and interact with molecules present on mucosal surfaces in the mouth, oropharynx, larynx, nasal cavity, or fauces. Alternatively, the bacterial strains do not attach or adhere to a mouth, oropharynx, larynx, nasal cavity, or fauces surface, but are present in these areas.

Examples of viridans Streptococci species that can be used in compositions of the invention include, but are not limited to S. sanguinis, S. parasanguinis, S. gordonii, S. oralis, S. uberis, S. mitis, S. rattus, S. salivarius, S. vestibularis, S. anginosus, S. constellatus, S. intermedius, S. mutans, S. sobrinus, and S. cricetus. Examples of Lactobacillus species that can be used in the compositions of the invention include, but are not limited to, L. acidophilus, L. jensenii, L. catenaforme, L. leichmanni, L. plantarum, L. johnsonii, L. gasseri, L. delbrueckii, L. casei, L. brevis, L. salivarius, L. sobrius, L. rhamnosus, L. reuteri, L. fermentum, L. paracasei, L. dextranicus, and L. helveticus. Examples of Bifidobacteria species that can be used in the compositions of the invention include, but are not limited to B. angulatum, B. animalis, B. asteroides, B. bifidum, B. bourn, B. breve, B. catenulatum, B. choerinum, B. coryneforme, B. cuniculi, B. dentium, B. gallicum, B. gallinarum, B indicum, B. longum, B. magnum, B. merycicum, B. minimum, B. pseudocatenulatum, B. pseudolongum, B. psychraerophilum, B. pullorum, B. ruminantium, B. saeculare, B. scardovii, B. simiae, B. subtile, B. thermacidophilum, and B. thermophilum. Examples of other non-pathogenic bacteria that can produce hydrogen peroxide include, without limitation, Pediococcus species, such as P. acidilactici, Leuconostoc species, such as L. mesenteroides, and Lactococcus species such as L. lactis.

The quantity of hydrogen peroxide produced by bacteria can be experimentally determined. See e.g. Hillman and Shivers, Arch. Oral. Biol., 33:395-401 (1988). The culture liquor of cells grown in the presence of oxygen is incubated with 40 μg/ml horseradish peroxidase and 0.4 μmol/ml o-dianisidine. After 2 minutes, the reaction is stopped by the addition of 0.02 ml of 5N HCl. The optical density of the sample is measured at 410 nm and the hydrogen peroxide concentration of the sample is calculated from a standard curve prepared using authentic hydrogen peroxide and an extinction coefficient at 230 nm of 81M⁻¹ cm⁻¹. In one embodiment of the invention one strain or species of bacteria or a combination of two or more strains or species of bacteria can produce at least about 0.01, 0.1, 0.5, 1, 2, 5 mM or more of H₂O₂ or any range or value between about 0.01 and about 5 mM.

In one embodiment of the invention a composition of the invention comprises one or more isolated Streptococcus oralis strains and/or one or more S. uberis strains. Compositions of the invention can comprise one or more isolated strains of S. oralis, for example, ATCC 35037, ATCC 55229, ATCC 700233, ATCC 700234, and ATCC 9811. Other strains of S. oralis include KJ3 and KJ3sm. KJ3sm is a naturally occurring genetic variant of KJ3 that is resistant to 1 mg/ml streptomycin sulfate. The streptomycin resistance is advantageous because it provides a marker for easy isolation and identification of the bacteria. Additionally, streptomycin resistant strains are slightly attenuated and do not survive as long in an oral cavity as wild-type strains. See Bammann et al., Infect. Immun. 22:721 (1978). This property is useful where the goal is to non-persistently colonize the mouth, oropharynx, fauces, larynx or nasal cavity of a subject with the bacteria.

Compositions of the invention can comprise one or more isolated strains of S. uberis, for example, ATCC 13386, ATCC 13387, ATCC 19435, ATCC 27958, ATCC 35648, ATCC 700407, ATCC 9927, strain KJ2 or strain KJ2sm. KJ2sm is a naturally occurring genetic variant of KJ2 that is resistant to 1 mg/ml streptomycin sulfate and provides the same advantages as for streptomycin-resistant strains of S. oralis. One or more isolated strains of S. oralis or one or more isolated strains of S. uberis, or both, can be used in compositions and methods of the invention.

Compositions of the invention can optionally comprise one or more isolated non-pathogenic mutans streptococcus species or strains, and/or one or more isolated non-pathogenic, hydrogen peroxide-producing Lactobacillus species or strains, viridans streptococcus species or strains, Bifidobacteria species or strains, one or more non-pathogenic Lactococcus species or strains, Pediococcus species or strains and/or one or more Leuconostoc species or strains deficient in the production of lactic acid. The mutans streptococcus species include, for example, S. rattus, S. cricetus, S. mutans, S. sobrinus, S. downeii, S. macacae, and S. ferus. A bacterium of the invention does not substantially produce L-(+)-lactate dehydrogenase (LDH). Such a strain is termed an LDH-deficient strain. An LDH-deficient strain produces 75%, 80%, 90%, 95%, 98%, 99%, or 100% less lactic acid than wild-type strains of the bacteria. An LDH-deficient strain can be a naturally occurring strain or a genetically modified strain. LDH-deficient bacteria can compete with and/or displace oral pathogenic bacteria such as S. mutans, a principal etiological agent of dental caries, in the oral cavity. LDH-deficient strains will compete with, for example, S. mutans for the same nutrients, colonization sites, etc. Therefore, LDH-deficient strains can be used to, for example, prevent and/or treat dental caries. LDH-deficient strains of bacteria of the invention are non-pathogenic, alter the microenvironment of the oral cavity to prevent colonization or outgrowth of pathogenic organisms, and/or displace pathogenic organisms from the oral cavity where the pathogen is part of the host's indigenous flora. The combination of non-pathogenic, hydrogen peroxide-producing bacteria and/or LDH-deficient bacteria provides a significant practical advantage in that the combination can used to prevent and treat, for example, respiratory infections and provide oral care benefits.

Examples of LDH-deficient mutans streptococcus strains include, for example, S. rattus JH145 (ATCC 31377) (a spontaneous, naturally-occurring LDH-deficient mutant) and JH140 (ATCC 31341) (a chemically-modified LDH-deficient mutant). See e.g., Stanshenko & Hillman, Microflora of plaque in rats following infection with an LDH-deficient mutant of Streptococcus rattus, Caries Res. 23:375-377 (1989); Hillman, Lactate dehydrogenase mutants of Streptococcus mutans: Isolation and preliminary characterization. Infect. Immun. 21:206-212 (1978); Abhyankar et al., Serotype c Streptococcus mutans mutatable to lactate dehydrogenase deficiency. J. Dent. Res. 64:1267-71 (1985); WO2005/018342; WO2009/152331.

The use of two, three, four, five, or more different species or strains of bacteria in compositions of the invention can provide an advantage over using fewer species or strains. This is because different species or strains of bacteria can colonize different surfaces or portions of the mouth, oropharynx, larynx, nasal cavity, or fauces. Therefore, the use of more than one species or strains of bacteria can be used to “blanket” all or most surfaces of the mouth, oropharynx, larynx, nasal cavity, or fauces, whereas the use of fewer species or strains of bacteria may result in certain surfaces or portions of the mouth, oropharynx, larynx, nasal cavity, or fauces being uncolonized or not populated by beneficial or commensal bacteria. Therefore, all or most surfaces of the mouth, oropharynx, larynx, nasal cavity, or fauces are exposed to the action of the bacteria of the invention.

Where two or more different species or strains of bacteria are used in compositions of the invention, each species or strain can be present in any amount as compared to the other species or strains (i.e., about 0.001% to about 99.999% by weight (or any range between about 0.001% to about 99.999%).

Bacteria of the invention can be obtained from sources such as humans or animals using isolation techniques well known to those of skill in the art. Alternatively, isolated bacteria can be obtained from biological resource centers, such as American Type Culture Collection (ATCC), P.O. Box 1549, Manassas, Va. 20108, USA.

The bacteria of the invention can be administered in the form viable bacteria or non-viable bacteria such as killed cultures. Killed cultures can include thermally killed bacteria, or bacteria killed by exposure to altered pH, subjected to pressure, or subject to other methods of killing.

In one embodiment of the invention, the bacteria of the invention maintain viability in the mouth, oropharynx, larynx, nasal cavity and/or fauces. It is not necessary for the bacteria of the invention to enter into or remain viable in the gastrointestinal tract. Additionally, administration of non-viable bacteria can induce temporary benefits. Where the bacteria are not viable, they are not able to grow and are not metabolically active, and are more limited in their ability to continuously deliver a beneficial effect. As a result, the host may need to be dosed regularly in order to maintain the health benefits. In contrast, viable bacteria that colonize or otherwise remain in the mouth, oropharynx, larynx, nasal cavity, and fauces are better able to deliver beneficial effects for a longer period of time.

Additionally, ³²P suicide-induced non-replicating bacterial cells of the invention can be used to provide beneficial effects. See e.g., U.S. Ser. No. 61/293,884, filed Jan. 11, 2010; WO2011/085367, filed Jan. 1, 2011. ³²P suicide-induced non-replicating bacterial cells are non-replicating bacteria of the invention that are metabolically active. That is, catabolism and anabolism occur in the bacteria, but the bacteria are substantially unable to replicate. The host may need to be dosed regularly in order to maintain health benefits.

Compositions of the invention can further comprise one or more carbon sources that are metabolizable by the one or more isolated, non-pathogenic, hydrogen peroxide-producing bacterial species or strains or the one or more lactate dehydrogenase deficient mutans Streptococcus species or strains or both types of species or strains. Carbon sources include, but are not limited to, for example, glucose, sorbitol, mannitol, fructose, galactose, maltose, sucrose, xylose, lactose, glycerol or combinations thereof.

Compositions of the invention can be combined with one or more additional ingredients such as vitamins (e.g., vitamin D, vitamin A, vitamin C, vitamin B1, B2, B3, B5, B6, B7, B9, B12, vitamin E), iron, iodine, zinc, copper, selenium, polyphenols, L-tryptophan, taurine, histidine, carnosine, alanine, cysteine, carotenoids, lutein, zeaxanthin, astaxanthin, bixin, lycopene, antioxidants, coenzymeQ10, decongestants, anticholinergics, analgesics, anti-inflammatories, antipyretics, antivirals, antitussives, expectorants, mucolytics, antihistamines, non-sedating antihistamines, local anesthetics, demulcents, sleep aids, and combinations thereof. When present, the compositions can comprise from about 0% to about 20%, alternatively from about 0.0001% to about 15%, alternatively from about 0.001% to about 10%, and alternatively from about 0.01% to about 5% of the additional ingredients by weight of the composition.

When present, the one or more additional ingredients can comprise from about 0.001 mg to about 1000 mg (or any range between about 0.001 mg to about 1000 mg), alternatively from about 2.5 mg to about 750 mg (or any range between about 2.5 mg to about 750 mg), and alternatively from about 5 mg to about 600 mg (or any range between about 5 mg to about 600 mg) of the one or more additional ingredients, per dose of the composition.

The compositions of the invention can comprise a pharmaceutically acceptable or nutritionally acceptable carrier. The carrier is physiologically compatible with the area of the subject to which it is administered. Carriers can be comprised of solid-based, dry materials for formulation into tablet, capsule, lozenge, or powdered form. A carrier can also be comprised of liquid or gel-based materials for formulations into liquid, gel, and chewing gum forms. The composition of the carrier can be varied so long as it does not interfere significantly with the therapeutic activity of the bacterial strains of the invention. A carrier can be a sugar alcohol such as erythritol, lactitol, maltitol, mannitol, sorbitol, and xylitol.

A composition can be formulated to be suitable for oral administration in a variety of ways, for example in a solid, semi-solid, liquid (including, e.g., a viscous liquid, a paste, a gel, or a solution), a dried mass, a dentifrice, a mouth wash, an oral rinse, a liquid suspension, a beverage, a topical agent, a powdered food supplement, a paste, a gel, a solid food, an oral rinse, a packaged food, a wafer, lozenge, chewing gum and the like. Other formulations will be readily apparent to one skilled in the art. A composition of the invention can include a nutrient supplement component and can include any of a variety of nutritional agents, as are well known, including vitamins, minerals, essential and non-essential amino acids, carbohydrates, lipids, foodstuffs, dietary supplements, and the like.

Compositions of the invention can also include natural or synthetic flavorings and food-quality coloring agents, all of which are compatible with maintaining viability of the bacterial species or strains of the invention.

A composition of the invention can include one or more gelling agents that can act as an adhesive agent to adhere the composition to the teeth, mouth, oropharynx, larynx, nasal cavity, or fauces. The concentration of the gelling agent may be greater than about 2, 4, 6, 8, 10, 15, 20, 30, 40, 50, 60, 70, 80 or less than about 80, 70, 60, 50, 40, 30, or 20 percent by weight of the composition.

Suitable gelling agents and adhesion agents useful in the present invention include, for example, silicone, polyethylene oxide, polyvinyl alcohol, polyalkyl vinyl ether-maleic acid copolymer (PVM/MA copolymer) such as, Gantrez AN 119, AN 139, and S-97, polyvinyl alcohol, polyacrylic acid, Poloxamer 407 (Pluronic), polyvinyl pyrrolidone-vinyl acetate copolymer (PVP/VA copolymer), such as Luviskol VA, and Plasdone S PVP/VA, polyvinyl pyrrolidone (PVP, e.g., K-15 to K-120), Polyquaterium-11 (Gafquat 755N), Polyquaterium-39 (Merquat plus 3330), carbomer or carboxypolymethylene (Carbopol), hydroxypropyl methylcellulose, hydroxyethyl cellulose, hydroxypropyl cellulose, corn starch, carboxymethyl cellulose, gelatin and alginate salt such as sodium alginate, natural gums such as gum karaya, xanthan gum, Guar gum, gum arabic, gum tragacanth, and mixtures thereof.

A humectant or plasticizer can be present in compositions of the invention. Humectants or plasticizers include, for example, glycerin, glycerol, sorbitol, polyethylene glycol, propylene glycol, and other edible polyhydric alcohols. The humectants or plasticizers can be present between at about 1% to about 99%, about 10% to about 95%, or at between about 50% and about 80% (or any range between 1% and 99%) by weight of a composition.

Bacteria of the invention can be prepared in, for example, a fermenter. The bacteria can be harvested from the fermenter and can be, for example, concentrated. Bacteria of the invention can be prepared for use by, for example, dehydration, air drying, lyophilizing, freezing, spray-drying. Bacteria can also be prepared for use by microencapsulation (see e.g., U.S. Pat. No. 6,251,478) or by coating with a protective substance such as, for example, lipid material such as triacylglycerols, waxes, organic esters, soybean oil, cottonseed oil, palm kernel oil, and esters of long-chain fatty acids and alcohols. In one embodiment of the invention the coated or encapsulated bacteria of the invention are released in the mouth, oropharynx, larynx, nasal cavity, and/or fauces of the host.

### Methods of Treatment and Prevention of Respiratory Infections

The bacterial species or strains can be present in a composition of the invention in a therapeutically effective amount. Therapeutically effective means effective to alleviate, reduce severity (e.g., 5, 10, 20, 30, 40, 50, 60, 70, 80, 90 or 100% less severe than controls that did not receive the composition), reduce duration (e.g., 5, 10, 20, 30, 40, 50, 60, 70, 80, 90 or 100% shorter duration than controls that did not receive the composition), reduce the number (e.g., 5, 10, 20, 30, 40, 50, 60, 70, 80, 90 or 100% few respiratory infections than controls that did not receive the composition), prevent, and/or ameliorate one or more symptoms (e.g., 5, 10, 20, 30, 40, 50, 60, 70, 80, 90 or 100% less symptoms or less severe symptoms than controls that did not receive the composition) of one or more respiratory infections. Therapeutically effective also can mean to eliminate or reduce the severity or reduce the duration of a respiratory infection.

A therapeutically effective amount or dosage is an amount or dosage of a composition of the invention at high enough levels to significantly improve the condition to be prevented and/or treated, but low enough to avoid serious side effects (at a reasonable benefit/risk ratio), within the scope of sound medical/dental judgment. The therapeutically effective amount or dosage of a composition of the invention may vary with the particular condition being treated, the age and physical condition of the patient being treated, the severity of the condition, the duration of treatment, the nature of concurrent therapy, the specific form of the source employed, and the particular vehicle from which the composition is applied.

The compositions of the invention can be applied in a therapeutically effective amount to the mouth, oropharynx, larynx, nasal cavity, and/or fauces for the treatment and/or prevention of one or more respiratory infections. A composition of the invention may be swallowed or may be rinsed around the oral cavity and then spit out, such that it is not substantially delivered to the gastrointestinal tract. That is, less than about 10, 5, 4, 3, 2, or 1, 0.5, or 0.1% (or any range or value between about 10 and 0.1%) of the delivered bacteria are delivered to the gastrointestinal tract. Treatment means inducing a reduction in the amount, type, or intensity or duration (or combination thereof) of one or more symptoms of a respiratory infection.

Prevention means that substantially no respiratory infection symptoms occur after exposure of the host to one or more pathogens that can cause respiratory infections either permanently (as long as the bacteria of the invention remain in sufficient numbers in the subject's mouth, oropharynx, larynx, nasal cavity and/or fauces), or temporarily (e.g., for about 1, 2, 5, 10 or more days or for about 1, 2, 3, 4, 5, 6 or more months).

In one embodiment of the invention prevention means prevention in a population of subjects. That is, given a population of subjects, the treatment can prevent respiratory infections in about 5, 10, 20, 30, 40, 50, 60, 70, 80, 90% or less of the subjects as compared to a control population that did not receive the treatment.

The bacterial strains of the invention can form at least a part of the transient or indigenous flora of the mouth, oropharynx, larynx, nasal cavity, and/or fauces and exhibit beneficial prophylactic and/or therapeutic effects in the respiratory tract. Additional oral care benefits of compositions of the invention include, for example, the treatment and/or prevention of dental caries, periodontitis, oral bacterial infections and diseases, oral wounds, Candida or fungal overgrowth, halitosis, or xerostomia-induced dental caries and associated periodontal diseases, the promotion of wound healing, teeth whitening or a combination thereof to a subject.

One embodiment of the invention provides a method for treating a respiratory infection comprising administering a composition comprising one or more isolated, non-pathogenic, hydrogen peroxide-producing bacterial species or strains to mouth, oropharynx, larynx, nasal cavity, fauces or combination thereof to a subject in need thereof. That is, the subject has one or more respiratory infections.

One embodiment of the invention provides for the treatment and/or prevention of respiratory infections in normal, healthy subjects. In another embodiment of the invention provides for treatment and/or prevention of respiratory infections in subjects having an increased susceptibility to respiratory infections as compared to normal, healthy subjects. In both embodiments, the method consists of administering a composition comprising one or more isolated, non-pathogenic, hydrogen peroxide-producing bacterial species or strains to a mouth, oropharynx, fauces, larynx, nasal cavity, or combination thereof to the subject. The composition can further comprise one or more lactate dehydrogenase-deficient mutans Streptococcus species or strains.

Subjects have an increased susceptibility to respiratory infection when they are more likely than a normal, healthy host to acquire a respiratory infection. Such hosts may have, for example, asthma, cystic fibrosis, mesothelioma, emphysema, chronic obstructive pulmonary disorder, acute respiratory distress syndrome, obstructive lung disease, chronic obstructive pulmonary disease, restrictive lung disease, any other lung disease, malignant or benign tumors of the respiratory system, are smokers, or are infants (0 to 2 years old or 6 months to 2 years old), children (3 years to 18 years old), or elderly (older than 65).

The invention also provides a method of reducing the amount in a subject of bacteria, fungi, or viruses that can cause a respiratory infection. The method comprises administering a composition comprising one or more isolated, non-pathogenic, hydrogen peroxide-producing bacterial species or strains to a mouth, oropharynx, fauces, larynx or nasal cavity or a combination thereof of a subject having one or more strains or species of bacteria, viruses or fungi that can cause a respiratory infection in their mouth, oropharynx, fauces, larynx or nasal cavity. The compositions can be administered on regular or daily basis. The number of one or more strains or species of bacteria, viruses or fungi that can cause a respiratory infection in the subject is reduced. The reduction can be about a 5, 10, 25, 50, 75, 90, 95, 99, or 100% (or any range between about 5% and about 100%) reduction in numbers. The composition can further comprise one or more lactate dehydrogenase-deficient mutans Streptococcus species or strains. Optionally, prior to the administration of the composition of the invention, one or more bacteria, viruses, or fungi that can cause a respiratory infection can be detected using any detection method known in the art. Those of skill in the art are aware of methods of detection of bacteria, viruses, and fungi that cause respiratory infections. Optionally, prior to the administration of the composition of the invention, one or more respiratory infections can be diagnosed in the subject using any methodology known in the art. Those of skill in the art are aware of methods of diagnosis of bacteria, viruses, and fungi that cause respiratory infections. Diagnosis is the identification of the cause or likely cause of a respiratory infection.

Another embodiment of the invention provides a method of preventing a respiratory infection or one or more symptoms of a respiratory infection in a subject. The method comprises obtaining data regarding a therapeutically effective dosage range for prevention of one or more respiratory infection symptoms in a particular type of subject and determining the effective dosage range for the particular type of subject. A particular type of subject can be, for example, a subject with asthma, cystic fibrosis, mesothelioma, emphysema, chronic obstructive pulmonary disorder or acute respiratory distress syndrome, obstructive lung disease, chronic obstructive pulmonary disease, restrictive lung disease, any other lung disease, malignant or benign of the respiratory system, are smokers, or are infants (0 to 2 years old or 6 months to 2 years old), children (3 years to 18 years old), an adult, or elderly (older than 65). The determined therapeutically effective dosage range for the particular type of subject of one or more isolated, non-pathogenic, hydrogen peroxide-producing bacterial species or strains is administered to a mouth, oropharynx, larynx, nasal cavity, fauces, or combination thereof of the particular type of subject. The administered composition can further comprise one or more lactate dehydrogenase-deficient mutans Streptococcus species or strains.

Compositions can be administered to the mouth, oropharynx, larynx, nasal cavity, and/or fauces of a host or subject such as an animal, including a mammal, for example, a human, a non-human primate, a dog, a cat, a horse, a bovine, a goat, or a rabbit.

The compositions of the invention can be orally administered in for example, food, water, a dentifrice, a gel, a paste, an emulsion, aerosol spray, chewing gum, lozenge, tablet, capsule, or a liquid suspension. The bacteria can either be already formulated into food, water, gel or other carrier or can be a composition (e.g., powder, tablet or capsule) that is added to the carrier (e.g., food, water, dentifrice, gel, paste, emulsion, aerosol spray, or liquid suspension) by the user prior to consumption.

One embodiment of the invention provides a method of non-persistently colonizing an oral cavity of a subject with therapeutically-effective bacteria comprising administering to the oral cavity of a subject a composition of the invention. In one embodiment of the invention the administered bacterial strains do not permanently colonize the mouth, oropharynx, larynx, nasal cavity, and/or fauces, rather the strains are present in the oral cavity for about 1 day, about 1 week, about 2 weeks, about 3 weeks, about 1 month, about 3 months or about 12 months after administration of the bacteria.

Compositions of the invention can be administered at a dose of about 1×10³, 1×10⁵, 1×10⁷, 1×10⁸, 1×10⁹, or 1×10¹¹ CFU (or any range or value between about 1×10³ and about 1×10¹¹) of viable bacteria. A dose of a composition of the invention can be administered at four times a day, three times a day, twice a day, once a day, every other day, two times a week, weekly, biweekly, or monthly. One, two, or more doses of a composition of the invention can be administered per day for about 1 week, about 2 weeks, about 1 month, about 2 months, about 3 months, about a year or more.

Compositions of the invention can also be administered by inhalative administration in the form of nasal sprays, nasal drops, nasal mists, nasal lavage, aerosol mixtures, quick-dissolving tablets, inhaled powders, oral inhalation solutions or suspensions, syrups, mechanized intermittent fluid pulsers, inhalers, respirators, transpirators, atomizers, vaporizers, air masks, insufflators, or nebulizers.

A composition of the invention can be applied to the mouth, oropharynx, larynx, nasal cavity, and/or fauces for between about 1 minute and about 8 hours. In some embodiments, the composition can be applied for greater than about 1, 2, 3, 4, 5, 6, 7, 8, 9, 10, 15, 20, 30, 40, 50, 60, 90, 120, 150, 180, 210, 240, 270, 300, 330, 360, 390, 420, 450, 480 minute(s) (or any range or value between about 1 and about 480 minute(s)) and/or less than 480, 450, 420, 390, 360, 330, 300, 270, 240, 210, 180, 150, 120, 90, 60, 50, 40, 30, 20, 15, 10, 9, 8, 7, 6, 5, 4, 3, 2 or 1 minute(s) (or any range or value between about 480 and about 1 minute(s)) and any combination thereof, wherein the bacterial species or strains have a concentration between about 0.01% and about 50%, or about 0.1% to about 25%, or about 1.0% to about 10% or any ranges or values in between 0.01% and 50% by weight of the composition. Such a regimen could be advantageously used once a day for greater than about one month, two months, four months, six months, twelve months, eighteen months, two years, five years, eight years, ten years, a lifetime, and/or less than about fifteen years, ten years, eight years, five years, two years, 18 months, 12 months, six months, four months, two months, one month and any combination thereof.

A kit of the invention can contain a one month, two month, three month, four month, five month, six month, or 12 month supply of a composition of the invention. A composition of the invention can be packaged and, in turn, a plurality of the packaged compositions can be provided in a storage container or outer package or carton.

All patents, patent applications, and other scientific or technical writings referred to anywhere herein are incorporated by reference in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

Human subjects used an oral breath mint composition comprising Streptococcus oralis KJ3, Streptococcus uberis KJ2, and Streptococcus rattus JH145 once a day for an average of between about 3 and 4 months. One hundred twenty six users of the composition were asked in a questionnaire if they felt that they had experienced fewer seasonal illnesses the past winter, such as colds or flu, while using the composition. Eighty six of the 126 users reported that they had experienced fewer seasonal illnesses the past winter including colds or flu, while using the composition. The same users were asked if they had any colds or flu in the past winter. If so, they were asked if the cold or flu symptoms were less severe while they were using the composition in comparison to past winters when the composition was not used. Seventy out of 114 users indicated that any cold or flu symptoms were less severe while using the composition than in the past when they were not using the composition.

I claim:

1. A method for treating a respiratory infection comprising administering a composition comprising one or more isolated, non-pathogenic, hydrogen peroxide-producing bacterial species or strains to mouth, oropharynx, larynx, nasal cavity, fauces or combination thereof to a subject in need thereof.

2. The method of claim 1, wherein the one or more isolated, non-pathogenic, hydrogen peroxide-producing bacterial species or strains are Lactobacillus, Bifidobacteria, viridans Streptococcus, Leuconostoc, Pediococcus, Lactococcus, or combinations thereof.

3. The method of claim 1, wherein the one or more isolated, non-pathogenic, hydrogen peroxide-producing bacterial species or strains are (a) one or more isolated Streptococcus oralis strains, or (b) one or more isolated strains of Streptococcus uberis, or (c) a combination thereof.

4. The method of claim 1, wherein the composition is not swallowed and not substantially delivered to the gastrointestinal tract.

5. The method of claim 3, wherein the composition is swallowed.

6. The method of claim 1, wherein the composition is administered to the subject up to four times a day, about once a week, or about once a month.

7. The method of claim 1, wherein the respiratory infection is reduced in severity or is eliminated, the symptoms of the respiratory infection are eliminated or reduced in severity or number, the duration of the respiratory infection is reduced in length of time, or a combination thereof.

8. The method of claim 1, wherein the composition further comprises one or more lactate dehydrogenase-deficient mutans Streptococcus, Lactobacillus, Bifidobacteria, viridans Streptococcus, Leuconostoc, Pediococcus, or Lactococcus species or strains.

9. The method of claim 8, wherein the one or more lactate dehydrogenase-deficient mutans Streptococcus species comprise Streptococcus rattus.

10. The method of claim 1, wherein the subject has increased susceptibility to a respiratory infection.

11. A method of reducing the amount in a subject of bacteria, fungi, or viruses that can cause a respiratory infection in a subject, comprising administering a composition comprising one or more isolated, non-pathogenic, hydrogen peroxide-producing bacterial species or strains to a mouth, oropharynx, fauces, larynx or nasal cavity or a combination thereof to a subject having one or more strains or species of bacteria, viruses or fungi that can cause a respiratory infection in their mouth, oropharynx, fauces, larynx or nasal cavity.

12. The method of claim 11, wherein the composition further comprises one or more lactate dehydrogenase-deficient mutans Streptococcus, Lactobacillus, Bifidobacteria, viridans Streptococcus, Leuconostoc, Pediococcus, or Lactococcus species or strains.

13. The method of claim 11, comprising, prior to the administration, detecting in the subject one or more bacteria, viruses, or fungi that can cause a respiratory infection.

14. The method of claim 11, comprising, prior to the administration, diagnosing in the subject a respiratory infection caused by one or more bacteria, viruses, or fungi.

15. A method of preventing a respiratory infection or one or more symptoms of a respiratory infection in a subject having an increased susceptibility to a respiratory infection comprising administering a composition comprising one or more isolated, non-pathogenic, hydrogen peroxide-producing bacterial species or strains to a mouth, oropharynx, fauces, larynx, nasal cavity, or combination thereof to the subject.

16. The method of claim 15, wherein the composition further comprises one or more lactate dehydrogenase-deficient mutans Streptococcus, Lactobacillus, Bifidobacteria, viridans Streptococcus, Leuconostoc, Pediococcus, or Lactococcus species or strains.

17. The method of claim 15, wherein the subject has cystic fibrosis, asthma, cystic fibrosis, emphysema, mesothelioma, chronic obstructive pulmonary disorder, acute respiratory distress syndrome, obstructive lung disease, chronic obstructive pulmonary disease, restrictive lung disease, malignant or benign tumors of the respiratory system, the subject is a smoker, or the subject is an infant or a child.

18. A method of preventing a respiratory infection or one or more symptoms of a respiratory infection in a subject comprising:
(a) obtaining data regarding an therapeutically effective dosage range for prevention of one or more respiratory infection symptoms in a particular type of subject and determining the therapeutically effective dosage range for the particular type of subject; and
(b) administering the therapeutically effective dosage range for the particular type of subject of one or more isolated, non-pathogenic, hydrogen peroxide-producing bacterial species or strains to a mouth, oropharynx, larynx, nasal cavity, fauces, or combination thereof to the particular type of subject.

19. The method of claim 18, wherein the particular type of subject is a subject with cystic fibrosis, an immune deficiency, asthma, cystic fibrosis, emphysema, mesothelioma, chronic obstructive pulmonary disorder, acute respiratory distress syndrome, obstructive lung disease, chronic obstructive pulmonary disease, restrictive lung disease, malignant or benign tumors of the respiratory system, the subject is a smoker, or the subject is an infant, a child, an adult or elderly.

20. The method of claim 18, wherein the composition further comprises one or more lactate dehydrogenase-deficient mutans Streptococcus, Lactobacillus, Bifidobacteria, viridans Streptococcus, Leuconostoc, Pediococcus, or Lactococcus species or strains.
